(19) United States
(12) Patent Application Publication
(10) Pub. No.: US 2016/0101228 A1
(43) Pub. Date: Apr. 14, 2016

(54) METHOD AND SYSTEM PROVIDING MORE ACCURATE FLUID TEMPERATURE MONITORING WITH SELECTABLE FLUID INPUT AND OUTPUT ARRAGEMENTS FOR BODY CAVITY TREATMENTS

(71) Applicants: John J. Landy, III, Billerica, MA (US); George Brussard, Lowell, MA (US)

(72) Inventors: John J. Landy, III, Billerica, MA (US); George Brussard, Lowell, MA (US)

(21) Appl. No.: 14/121,749

(22) Filed: Oct. 14, 2014

(51) Int. Cl. A61M 1/28; A61F 7/12; A61M 5/44; A61F 7/00; A61M 25/00

(52) U.S. Cl. CPC A61M 1/285; A61F 7/0085; A61M 25/0043; A61M 5/44; A61F 7/12; A61F 2007/0095; A61F 2007/0069; A61M 2205/3368; A61F 2007/126; A61M 2205/127

(57) ABSTRACT

Arrangements of components for use in support of a Hyperthermic Intraperitoneal Chemotherapy (HIPEC) System which conveys fluids from a system to a patient and back to the system in a complete circuit. The described arrangements provide multiple means of rapidly configuring the number of input lines and output lines into and out of the patient. Included are integral temperature probes in the inflow and outflow lines. Also included are various means of introducing and removing the fluids from the patient and additional temperature probes to be used at the discretion of the clinician.

The following described inventions are readily and conveniently usable with Hyperthermic Intraperitoneal Chemotherapy (HIPEC) Systems such as those described in co-pending U.S. patent application Ser. No. 12/924,920 filed Oct. 9, 2010; which is a Division of U.S. Pat. No. 7,819,835 assigned to the assignee of this patent application.

### BACKGROUND TO THE INVENTION

In hyperthermic lavage heated fluids are pumped into a patient's body cavity. The heated fluids deliver heat to the patient's body cavity and surrounding tissues in an effort to supply certain therapies. A cancer treatment that involves surgically removing cancerous tumors from the abdominal cavity; and then bathing the abdominal cavity with heated fluids to kill any cancer cells left in the abdominal cavity, is one of such therapies being performed. Such a treatment contemplates that heat at 42 degrees Celsius can destroy cancer cells without affecting normal cells in the body.

The placement of temperature probes within the fluid path at unspecified locations, for fluid input to the patient and for the outflow from the patient, have met with problems. It is generally difficult to predict the actual fluid path prior to the commencement of fluid flow. The result is that the temperature probes are not reliably placed within the fluid path; resulting in temperature measurements that are not representative of the heated chemotherapy but, instead may be closer to the temperature of the patient's circulatory system.

There are also concerns as to the number of fluid flow lines transitioning between the system and the patient. There are, as a minimum, one line to the patient, one line from the patient, and two temperature probes. Four lines crossing into a sterile field

There are also concerns regarding the return line from the patient. In order to initiate flow from the patient a negative pressure is required. This is usually accomplished by using a pump or the application of a vacuum. In practice both generate a low pressure that will pull fluids into the tubing and back to the system. The concern is that this low pressure can tear delicate organs if the tubing were to contact them and form a seal.

### SUMMARY OF THE INVENTION

It is therefore an object of this invention to provide new and novel systems for implementing hyperthermia.

It is another object of this invention to provide new and novel methods for implementing hyperthermia.

It is yet another object of this invention to provide new and novel apparatus for incorporation into hyperthermia implementation systems and methods.

It is yet another object of this invention to provide new and novel apparatus for incorporation into hyperthermia implementation systems and methods to be able to tune the fluid paths for each patient to best treat that particular patient by using different tubing sets with varying numbers of inputs and outputs.

It is yet still another object of this invention to provide new and novel hyperthermia systems to be used in therapeutic procedures in which fluid, at a few degrees centigrade above normothermic, is to be circulated in a fluid flow path: into a body cavity; withdrawn from the body cavity; reheated and recirculated; according to the requirements of the respective treatment(s).

It is still another object of this invention to provide integral temperature probes within the inflow and outflow tubing both prior to and following the inflow and outflow bifurcations resulting in a measurement of all of the multiple flow paths.

It is yet still another object of this invention to provide new and novel hyperthermia systems and methods, to be used in therapeutic procedures, in which the temperature of the flowing fluid are optimized and may be operation set.

It is yet still another object of this invention to provide new and novel hyperthermia systems and methods, to be used in therapeutic procedures by heating the therapeutic fluid to only a few degrees C., above normothermic, by circulating, withdrawing and re-circulating such fluid, to and through a body cavity, and by sensing the selected temperature while outside the body cavity; to provide an efficient hyperthermia system and method which is relatively effective and safe for the patient, and may be operator set.

It is yet still another object of this invention to provide new and novel hyperthermia systems and methods, to be used in therapeutic procedures by heating the therapeutic fluid to only a few degrees C., above normothermic, by circulating, withdrawing and re-circulating such fluid, to and through a body cavity, and by sensing the selected temperature prior to entry into the body cavity and following exit from the body cavity.

Other objects of this invention will hereinafter become obvious from the following description of the preferred embodiments of this invention.

### BRIEF DESCRIPTION OF THE DRAWINGS

In the drawings:

FIG. 1 is a schematic of a single lumen fluid flow line configuration particularly for a hyperthermia system and method, incorporating and embodying the principals of and the instant invention.

FIG. 2 is a schematic of a straight fluid flow director incorporating the instant invention

FIG. 3 is a schematic of a combination of fluid flow direction components for providing two fluid flow lines incorporating the instant invention.

FIG. 4 is an alternate embodiment of a schematic of a combination of fluid flow direction components for providing two fluid flow lines incorporating the instant invention.

FIG. 5 is a schematic of an alternate embodiment of a straight fluid flow director incorporating the instant invention

FIGS. 6 & 7 are schematics of alternate embodiments of fluid flow direction components for combining together with the combination of components of FIG. 4 to provide four fluid flow lines incorporating the instant invention.

FIGS. 8-10 are schematics of fluid flow direction components for combining together to provide further alternate arrangements of fluid flow lines incorporating the instant invention.

FIGS. 11-13 are schematics of fluid flow direction components for combining together to provide yet another alternate arrangements of fluid flow lines incorporating the instant invention.

FIG. 14 shows a schematic of a Christmas tree straight component of the instant invention. and

FIG. 15 shows a schematic of a Christmas tree “Y” component of the instant invention.

### DESCRIPTION OF THE INVENTIVE EMBODIMENTS

The subject matters of the instant inventions are directed to a hyperthermia process for an anatomical portion of a body. Therapeutic infusion fluids (not shown) are usually provided to such a process through either one or more conventionally available IV type bags or reservoir(s) and then to an infusion fluid disposable set, to direct the therapeutic fluid to a fluid processing device which is usually a device to heat the fluid to a given temperature. The therapeutic fluid after being processed by the device is directed into a patient tubular fluid feed line to a respective anatomical portion of a patient's body and then, from that anatomical portion, by a patient tubular fluid return line back to the respective reservoirs.

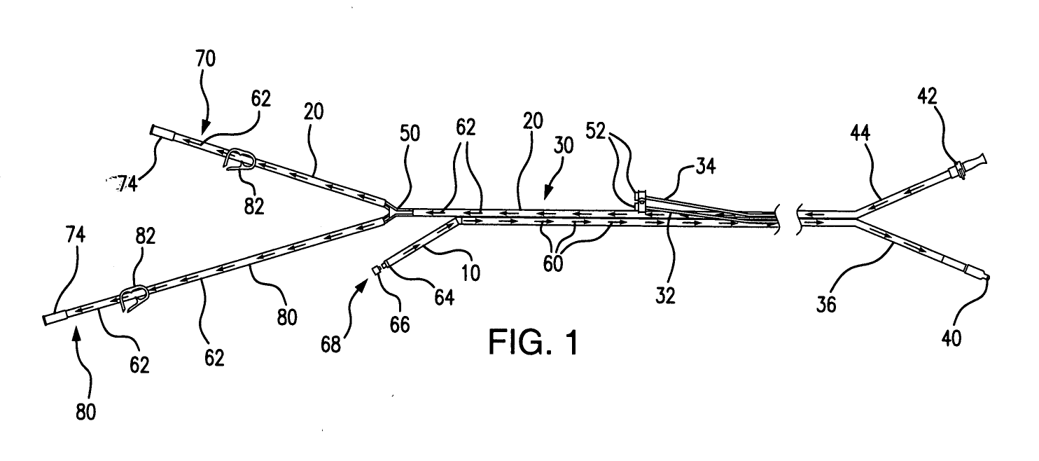

A fluid inlet tube or line 10 (FIG. 1) and a fluid circulation and outlet tube or line 20 (FIG. 1) are fused together into a single dual lumen line configuration 30. Additionally each lumen line 30 has an integral temperature probe placed within the fluid path to ensure that there is a temperature probe 32 in the inlet tubing 10 and a temperature probe 34 in the outlet tubing 20; thus eliminating the need to individually place such temperature probes within the patient. This results in a single line 30 crossing into the sterile field. Each line has at the patient end quick disconnects, a male quick connector 40 at the patient inflow end 36 and a female quick connect 42 at the patient outflow end 44, that allow rapid configuring of the input and output line configurations. Also provided is a bifurcated “Y” set 50 that provides two output lines (paths) thus allowing the number of output fluid paths to be adjusted. The arrangement of the components also includes protective sheaths with a multiplicity of holes such as Viaguards to protect the organs and introductory sumps or drains with a multiplicity of holes such as Medtronic sumps used to introduce fluid into the patient

It should be noted that temperature probes 32 and 34 (FIG. 1) are of the MERS 4400 Series type; but that other suitable temperature probes may just as well be used. Each temperature probe 32, 34 includes a sensing portion embedded in its respective tube (10, 20) and extends and is sealed as it passes through the wall of its respective tube and is fitted with a connector 52 whereat it can be connected to recording and/or a temperature indication device.

It should be further noted that respective fluid flow tubes 10, 20 are provided with inflow arrows 60 and outflow arrows 62 respectively to facilitate use of dual lumen 30. In addition a barbed female luir lock 64 with a vented male cap 66 provide a fluid entry port 68 for lumen 30.

The fluid exit flow path extends through tube 20 and includes two possible paths; a first path 70 terminating at an LDPE 74 cap is connectable to fluid treatment devices such as those which facilitate heating and fluid flow; while alternate path 80, which also terminates at an LDPE cap 74 is utilized to direct the fluid to a fluid disposal. It should be noted that each fluid flow path is provided with a pinch clamp 80.

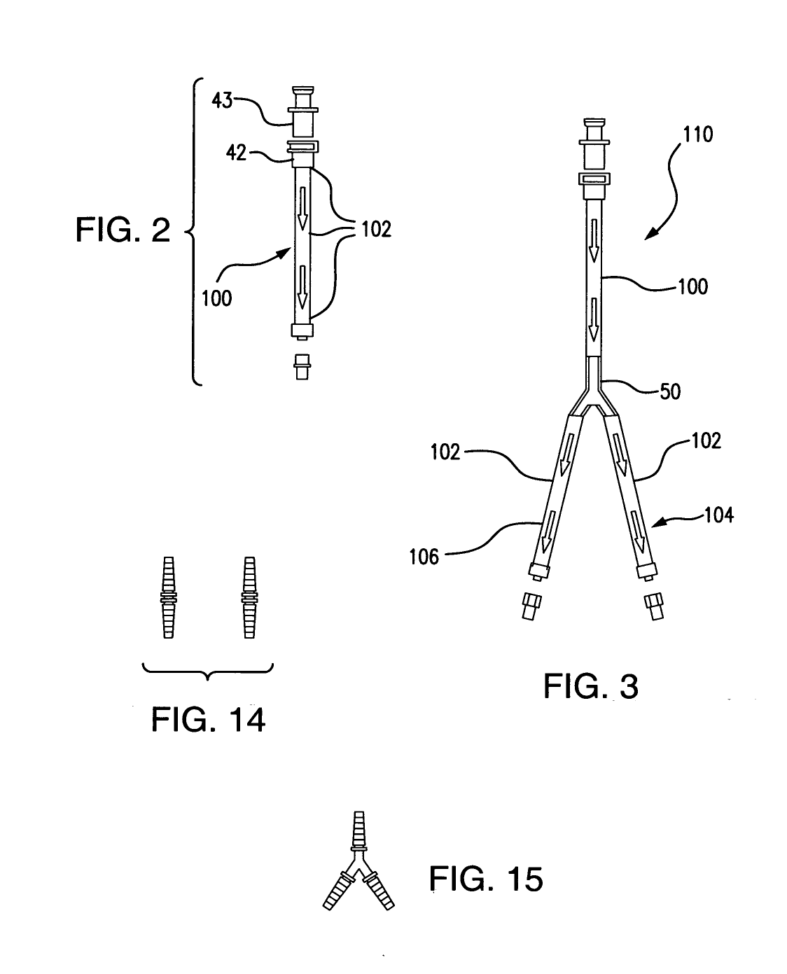

FIGS. 2-7 illustrate various arrangements of subject components of these inventions.

For example FIG. 2 is directed to a straight flow director 100 assembled with a female quick connector 42, a plug 43, and a vented female cap 101; all assembled with an output tube 102 to provide straight flow director 100.

A dual flow director 110 (FIG. 3) can be readily and easily assembled by attaching a “Y” 50 onto tube 100 and affixing tubes 102 as shown in FIG. 3 thus providing two fluid flow paths 104, 106 through tubes 102.

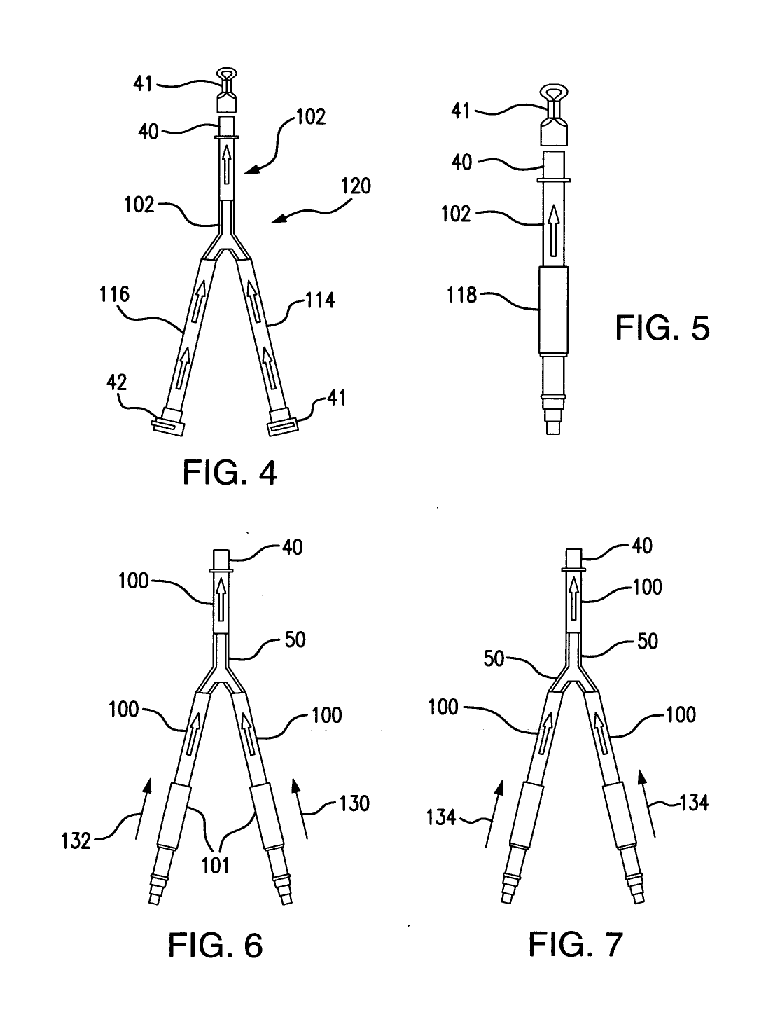

A flow director 120 may be readily and easy to assemble as shown, for example, in FIGS. 4 and 5. Flow director 120 utilizes a straight flow director 102 attached by a “Y” set 50 to two straight flow directors 114, 116 respectively. A male quick connector 40 is attached to tube 100 with a female cap quick connector 41 attached to the male quick connector. A female quick connector 42 is attached at the end of flow directors 114, 116 respectively. It should be noted that straight connector 102 (FIG. 5) includes a tube 118 and a perfusion adapter 119.

Additional fluid flow arrangements, either multiples of the arrangement of FIGS. 2 and 3, and/or the arrangement of FIGS. 4 and 5, or other combinations thereof, may be attachable, by way of example, to either flow path 114 or 116 or both of them as it would be by combining the respective arrangement of FIG. 4 with the arrangements of FIG. 5 and FIG. 6.; thus providing 2, 3 or 4 fluid flow paths such as: fluid flow paths 130, 132 (FIG. 6) to flow directors 114 (FIG. 4); and fluid flow paths 134, 136 (FIG. 7) to flow director 116 (FIG. 4)

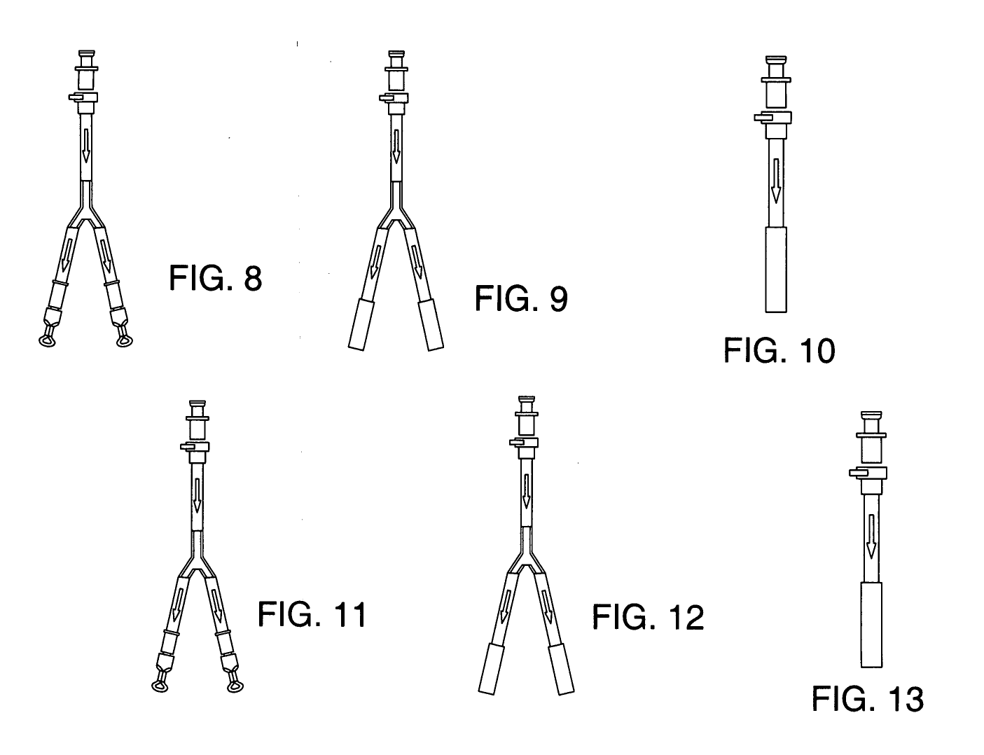

The fluid flow directors of FIGS. 8-10, 9-13, are readily and easily assembled as shown in their respective drawings. The directions of the fluid flow direction arrows in the respective arrangements should be kept in mind and consistent depending on whether the fluid flow arrangement is to direct fluid to the patient or out from the patient.

FIGS. 14 and 15 show Christmas tree straight 200 and Christmas tree “Y” 202 components fabricated so as to be able to adjust their respective ends.

The above described arrangements incorporate: dual lumen tubing (lines) with integral temperature probes and general purpose couplings; and may as well include modular inflow and outflow bifurcations capable to be configured in a number of ways with general purpose couplings; also SurgiMark Viaguards, Medtronic Sumps and additional Measurement Specialties temperature probes.

The above described components contacting the fluid path may be easily formed by extrusion or by welding or sealing tubing components, and should be sterilizable by conventional means.

What is claimed is:

1. A process for hyperthermia treatment for an anatomical portion of a body: comprising,
(a) providing treatment fluid;
(c) causing said treatment fluid to flow to the anatomical portion of the body to be treated and from that body portion;
(d) causing the fluid to flow from the body portion to be treated in a continuous enclosed path to and from the body to be treated until the treatment procedure is terminated; and
(e) controlling selected aspects of the process by continuously monitoring the fluid temperature prior to entry of the flowing treatment fluid into the body portion to be treated and following exit of the flowing treatment fluid from the body portion being so treated.

2. An arrangement of fluid flow lines to facilitate the flow of a fluid or fluids from a source of such fluids to a recipient to receive the fluids and from the recipient of the fluids; comprising,
(a) a first fluid flow line through which fluid is to flow through in a first given direction;
(b) a second fluid flow line through which fluid is to flow through in a second given direction; and
(c) a first temperature sensing device incorporated into said first fluid flow line to sense the temperature of fluid when flowing therethrough; and
(d) a second temperature sensing device incorporated into said second fluid flow line to sense the temperature of fluid when flowing therethrough.

3. The arrangement of fluid flow lines of claim 2 wherein said first fluid flow line and said second fluid flow line are secured to each other to form a single dual lumen line.

4. The arrangement of fluid flow lines of claim 2 wherein said first fluid flow line facilitates the flow of fluid in a first direction and said second fluid flow line facilitates the flow of fluid in a second direction, opposite to said first direction.

5. The arrangement of fluid flow lines of claim 2 including a selection of other fluid flow lines connectable to said first line or said second line to facilitate fluid flow in multiple directions.

6. The arrangement of fluid flow lines of claim 5, including, a sub-arrangement of fluid flow components including single direction flow tubes connected by Y configured flow through with said single direction flow tube including single direction flow through components connected to each leg of the Y.
